(19) United States
(12) Patent Application Publication
(10) Pub. No.: US 2012/0252768 A1
(43) Pub. Date: Oct. 4, 2012

(54) COMPOSITION CONTAINING PROTOPANAXATRIOL AND PROTOPANAXADIOL

(75) Inventors: Hideaki IWASAKI, Tokyo (JP); Mitsuru Nomura, Tokyo (JP); Naho Suzuki, Tokyo (JP); Hiroaki Kambayashi, Tokyo (JP); Kumiko Kitamura, Tokyo (JP)

(73) Assignee: LION CORPORATION, Tokyo (JP)

(21) Appl. No.: 13/495,031

(22) Filed: Jun. 13, 2012

Related U.S. Application Data
(63) Continuation of application No. PCT/JP2010/070708, filed on Nov. 19, 2010.

(30) Foreign Application Priority Data
Dec. 14, 2009 (JP) 2009-282764

(51) Int. Cl. A23L 1/30; A23L 2/38; A61P 29/00; A61K 31/58; A61P 35/00

(52) U.S. Cl. 514/170; 426/590; 426/648

(57) ABSTRACT

A composition including: at least one mixture selected from the group consisting of a mixture of (A) panaxatriol and (B) protopanaxatriol and a mixture of (C) panaxadiol and (D) protopanaxadiol, wherein a ratio (A)/(B) of a mass of the (A) panaxatriol to a mass of the (B) protopanaxatriol is 1 or greater, and a ratio (C)/(D) of a mass of the (C) panaxadiol to a mass of the (D) protopanaxadiol is 1 or greater.

### CROSS-REFERENCE TO RELATED APPLICATION

This is a continuation application of PCT/JP2010/070708, filed on Nov. 19, 2010.

### BACKGROUND OF THE INVENTION

1. Field of the Invention

The present invention relates to a composition containing at least one of protopanaxatriol and protopanaxadiol.

2. Description of the Related Art

Protopanaxatriol (PPT) and protopanaxadiol (PPD) are known to have various physiological activities such as an anti-cancer effect (see Japanese Patent Application Laid-Open (JP-A) Nos. 2005-504799 and 58-57399), an anti-inflammatory effect on skin diseases (see JP-A No. 2007-008896), an effect of suppressing excretion of urinary albumin (see JP-A No. 10-212296) and an effect of activating PPARγ which regulates the expression of genes playing important roles in fat metabolism and sugar metabolism (see Korean Patent Application Laid-Open No. 10-2006-0131012).

Protopanaxatriol (PPT) and protopanaxadiol (PPD) are aglycons remaining after removal of the sugar moiety from saponin (glycoside) (ginsenoside) contained in ginseng. They are compounds belonging to dammarane-type triterpenes.

Although protopanaxatriol (PPT) and protopanaxadiol (PPD) are white powder and insoluble to water, their solubility can be increased by the addition of an organic solvent.

The effect of saponin differs among individuals since saponin has low absorbability into bodies and its sugar is decomposed by intestinal bacteria before absorption. Meanwhile, such aglycons as protopanaxatriol (PPT) and protopanaxadiol (PPD) do not have sugar moieties, and thus can be expected to minimize the differences among individuals.

However, protopanaxatriol (PPT) and protopanaxadiol (PPD) each have an unstable structure and disadvantageously, they are decomposed minute by minute in a liquid system, especially in a low-pH system. Even in the powder form, they are progressively decomposed day by day at a temperature equal to or higher than ambient temperature. As described above, there is a problem that their stability is low.

Accordingly, at present, keen demand has arisen for provision of a more stable composition containing a large amount of at least one of protopanaxatriol (PPT) and protopanaxadiol (PPD) having physiological activities such as an anti-cancer effect, an anti-inflammatory effect and a sugar metabolism-regulating effect, and a highly safe food or beverage containing the composition.

### SUMMARY OF THE INVENTION

The present invention aims to solve the above existing problems and achieve the following objects. Specifically, an object of the present invention is to provide: a more stable composition containing a large amount of at least one of protopanaxatriol (PPT) and protopanaxadiol (PPD) having physiological activities such as an anti-cancer effect, an anti-inflammatory effect and a sugar metabolism-regulating effect; and a highly safe food or beverage containing the composition.

The present inventors conducted extensive studies to solve the above existing problems and have obtained the following findings. That is, they have found that mixing panaxatriol (PT) and protopanaxatriol (PPT) at a predetermined ratio improves stability of protopanaxatriol (PPT) and that mixing panaxadiol (PD) and protopanaxadiol (PPD) at a predetermined ratio improves stability of protopanaxadiol (PPD). The present invention has been accomplished on the basis of these findings.

The present invention is based on the above findings obtained by the present inventors. Means for solving the problems are as follows. Specifically, a composition of the present invention includes: at least one mixture selected from the group consisting of a mixture of (A) panaxatriol and (B) protopanaxatriol and a mixture of (C) panaxadiol and (D) protopanaxadiol, wherein a ratio (A)/(B) of a mass of the (A) panaxatriol to a mass of the (B) protopanaxatriol is 1 or greater, and a ratio (C)/(D) of a mass of the (C) panaxadiol to a mass of the (D) protopanaxadiol is 1 or greater.

The present invention can provide: a more stable composition containing a large amount of at least one of protopanaxatriol (PPT) and protopanaxadiol (PPD) having physiological activities such as an anti-cancer effect, an anti-inflammatory effect and a sugar metabolism-regulating effect; and a highly safe food or beverage containing the composition. These can solve the above existing problems and achieve the above objects.

### DETAILED DESCRIPTION OF THE INVENTION

### (Composition)

A composition of the present invention includes at least one mixture selected from the group consisting of a mixture of (A) panaxatriol and (B) protopanaxatriol and a mixture of (C) panaxadiol and (D) protopanaxadiol; and, if necessary, further includes other ingredients.

### <Ingredient (A)>

Panaxatriol (PT) (Ingredient (A)) is a compound having a structure expressed by the following Structural Formula (1). Panaxatriol (PT) is a compound belonging to dammarane-type triterpenes. It is an aglycon formed after the sugar moiety has been removed from a plant-origin saponin (glycoside) and then the side chain has been ring-closed.

The method for obtaining Ingredient (A) is not particularly limited and may be appropriately selected depending on the intended purpose. Examples thereof include a method in which Ingredient (A) is extracted from plants, a method in which Ingredient (A) is obtained through synthesis, and a method in which commercially available products of Ingredient (A) are used.

Incorporation of Ingredient (A) into the composition can advantageously stabilize Ingredient (B).

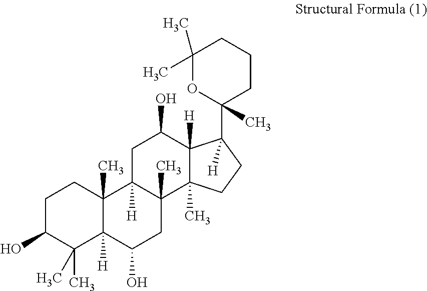

### <Ingredient (B)>

Protopanaxatriol (PPT) (Ingredient (B)) is a compound having a structure expressed by the following Structural Formula (2).

Protopanaxatriol (PPT) is a compound belonging to dammarane-type triterpenes. It is an aglycon formed after the sugar moiety has been removed from a plant-origin saponin (glycoside).

The method for obtaining Ingredient (B) is not particularly limited and may be appropriately selected depending on the intended purpose. Examples thereof include a method in which Ingredient (B) is extracted from plants, a method in which Ingredient (B) is obtained through synthesis, and a method in which commercially available products of Ingredient (B) are used.

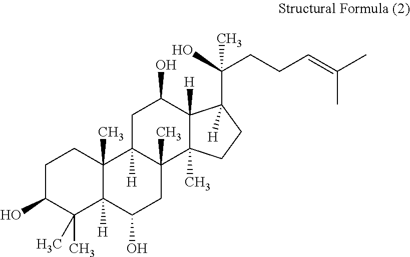

### <Ingredient (C)>

Panaxadiol (PD) (Ingredient (C)) is a compound having a structure expressed by the following Structural Formula (3).

Panaxadiol (PD) is a compound belonging to dammarane-type triterpenes. It is an aglycon formed after the sugar moiety has been removed from a plant-origin saponin (glycoside) and then the side chain has been ring-closed.

The method for obtaining Ingredient (C) is not particularly limited and may be appropriately selected depending on the intended purpose. Examples thereof include a method in which Ingredient (C) is extracted from plants, a method in which Ingredient (C) is obtained through synthesis, and a method in which commercially available products of Ingredient (C) are used.

Incorporation of Ingredient (C) into the composition can advantageously stabilize Ingredient (D).

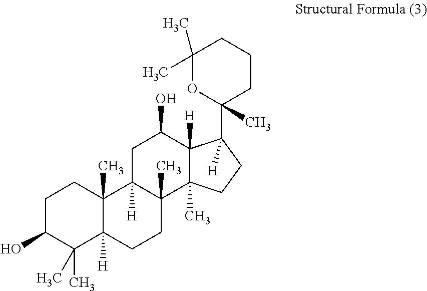

### <Ingredient (D)>

Protopanaxadiol (PPD) (Ingredient (D)) is a compound having a structure expressed by the following Structural Formula (4).

Protopanaxadiol (PPD) is a compound belonging to dammarane-type triterpenes. It is an aglycon formed after the sugar moiety has been removed from a plant-origin saponin (glycoside).

The method for obtaining Ingredient (D) is not particularly limited and may be appropriately selected depending on the intended purpose. Examples thereof include a method in which Ingredient (D) is extracted from plants, a method in which Ingredient (D) is obtained through synthesis, and a method in which commercially available products of Ingredient (D) are used.

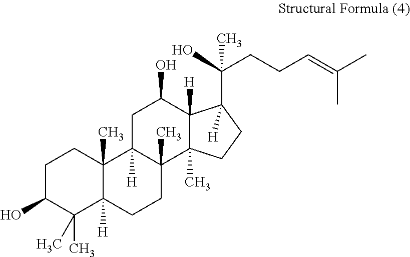

### <Amounts>

The amounts of Ingredients (A), (B), (C) and (D) contained in the composition are not particularly limited and may be appropriately selected depending on the intended purpose so long as the effects of the present invention are not impeded.

In the composition, Ingredient (A) is incorporated for stabilizing Ingredient (B) and Ingredient (C) is incorporated for stabilizing Ingredient (D). The composition may be a composition containing Ingredients (A) and (B) only among Ingredients (A), (B), (C) and (D), or may be a composition containing Ingredients (C) and (D) only among Ingredients (A), (B), (C) and (D). The composition may be a mixture of Ingredients (A) and (B) itself, or a mixture of Ingredients (C) and (D) itself.

### —Ratio by Mass of Ingredient (A) to Ingredient (B)—

The ratio by mass of Ingredient (A) to Ingredient (B); i.e., (A)/(B) is not particularly limited, so long as it is 1 or greater, and may be appropriately selected depending on the intended purpose. The ratio (A)/(B) is preferably 9 or greater, more preferably 19 or greater. When the ratio by mass of Ingredient (A) to Ingredient (B) falls within the above preferred range, the stability of protopanaxatriol (PPT) increases.

Notably, having the “stability” means that when Ingredient (B) (protopanaxatriol (PPT)) is in the form of powder, the residual rate of Ingredient (B) is 90% by mass or more after the composition is left to stand still for one month under conditions of 40° C. and relative humidity 75%.

Also, having the “stability” means that when Ingredient (B) (protopanaxatriol (PPT)) is in the form of ethanol solution, the residual rate of Ingredient (B) is 90% by mass or more after the composition is left to stand still for one month under conditions of 40° C. and relative humidity 75%.

### —Ratio by Mass of Ingredient (C) to Ingredient (D)—

The ratio by mass of Ingredient (C) to Ingredient (D); i.e., (C)/(D) is not particularly limited, so long as it is 1 or greater, and may be appropriately selected depending on the intended purpose. The ratio (C)/(D) is preferably 9 or greater, more preferably 19 or greater. When the ratio by mass of Ingredient (C) to Ingredient (D) falls within the above preferred range, the stability of protopanaxadiol (PPD) increases.

Notably, having the “stability” means that when Ingredient (D) (protopanaxadiol (PPD)) is in the form of powder, the residual rate of Ingredient (D) is 90% by mass or more after the composition has been left to stand still for one month under conditions of 40° C. and relative humidity 75%.

Also, having the “stability” means that when Ingredient (D) (protopanaxadiol (PPD)) is in the form of ethanol solution, the residual rate of Ingredient (D) is 90% by mass or more after the composition has been left to stand still for one month under conditions of 40° C. and relative humidity 75%.

### <Other Ingredients>

The other ingredients are not particularly limited and may be appropriately selected depending on the intended purpose. Examples thereof include supplemental materials or additives commonly used for the production of foods or beverages.

The supplemental materials or additives are not particularly limited and may be appropriately selected depending on the intended purpose. Examples thereof include glucose, fructose, sucrose, maltose, sorbitol, stevioside, rubusoside, corn syrup, lactose, citric acid, tartaric acid, malic acid, succinic acid, lactic acid, L-ascorbic acid, dl-α-tocopherol, sodium erythorbate, glycerin, propylene glycol, glycerin fatty acid esters, polyglycerin fatty acid esters, sucrose fatty acid esters, sorbitan fatty acid esters, gum arabic, carrageenan, casein, gelatin, pectin, agar, B vitamins, nicotinic-acid amide, calcium pantothenate, amino acids, calcium salts, dyes, perfumes and preservatives.

The amount of the other ingredients contained in the composition is not particularly limited and may be appropriately selected depending on the intended purpose.

### <Use>

The manner in which the composition is used is not particularly limited and may be appropriately selected depending on the intended purpose. The composition is preferably used as a food or beverage which can be given orally.

Here, “food or beverage” refers to those which are less harmful to human health and which are given orally or through the gastrointestinal tract in the ordinary social life. They are not limited to foods, drugs and quasi drugs within the administrative boundaries, but include a wide variety of orally-given common foods, healthy foods, health-promoting foods, quasi drugs and drugs.

The composition may be a food or beverage itself, or may be incorporated into a food or beverage.

The amount of the composition incorporated into a food or beverage is not particularly limited and may be appropriately selected depending on the type of the food or beverage so long as the effects of the present invention are not impeded.

### <Food or Beverage>

The food or beverage is not particularly limited and may be appropriately selected depending on the intended purpose so long as it contains at least one of a mixture of Ingredients (A) and (B) and a mixture of Ingredients (C) and (D). Examples thereof include beverages such as refreshing beverages, carbonated beverages, energy beverages, fruit beverages and lactic beverages; frozen desserts such as ice cream, ice sherbet and ice shavings; noodles such as buckwheat noodles, wheat noodles, vermicelli, coats of Chinese dumplings, coats of pork dumplings, Chinese noodles and instant noodles; snacks such as candies, gum, chocolate, tabletted snacks, munches, biscuits, jelly, jam, cream, baked confectionery and bread; marine products such as crab, salmon, Japanese littleneck, tuna, sardine, shrimps, prawns, bonito, mackerel, whale, oyster, saury, squid, bloody clam, scallop, abalone, sea chestnut, salmon caviar and Sulculus diversicolor supertexta; marine/livestock processed foods such as fish minced and steamed, ham and sausage; dairy products such as processed milk and fermented milk; fats and oils or processed foods thereof such as salad oil, Tempura oil, margarine, mayonnaise, shortening, whip cream and dressing; seasonings such as sauce and basting; retort pouch foods such as curry, stew, Oyako-don (a bowl of rice topped with boiled chicken and eggs), rice porridge, Zosui (rice soup), Chuka-don (a bowl of rice with a chop-suey-like mixture on it), Katsu-don (a rice bowl with pork cutlets), Ten-don (a tempura rice bowl), Una-don (an eel rice bowl), hayashi rice (hashed beef with rice), Oden (a dish containing several ingredients such as boiled eggs and radish), mapo doufu, Gyu-don (a beef rice bowl), meat sauce, egg soup, rice omelet, Chinese dumplings, pork dumplings, hamburger steak and meat balls; healthy foods in various forms and dietary supplements; and pharmaceutical drugs and quasi drugs such as tablets, capsules, drinkable preparations, jelly preparations and troches.

### <Dosage Form>

The dosage form of the food or beverage is not particularly limited and may be appropriately selected depending on the intended purpose. Examples thereof include an oral solid preparation, an oral semisolid preparation and an oral liquid preparation.

Ingredients (B) and (D) are generally poor in stability and thus are not suitable to use in a system containing a large amount of water and a low-pH system. However, Ingredients (B) and (D) in the composition of the present invention are stable even in a system containing a large amount of water and a low-pH system, and thus may be formed into an oral semisolid preparation and an oral liquid preparation.

### —Oral Solid Preparation—

The oral solid preparation is not particularly limited and may be appropriately selected depending on the intended purpose. Examples thereof include a tablet, a coated tablet, granules, powder and a capsule.

The method for producing the oral solid preparation is not particularly limited and may be a routine method. For example, the oral solid preparation can be produced by adding an excipient and, if necessary, various additives to at least one of a mixture of Ingredients (A) and (B) and a mixture of Ingredients (C) and (D). Here, the excipient is not particularly limited and may be appropriately selected depending on the intended purpose. Examples of the excipient include lactose, sucrose, sodium chloride, glucose, starch, calcium carbonate, kaolin, microcrystalline cellulose and silicic acid. The additives are not particularly limited and may be appropriately selected depending on the intended purpose. Examples of the additives include a binding agent, a disintegrating agent, a lubricating agent, a coloring agent and a sweetening/flavoring agent.

The binding agent is not particularly limited and may be appropriately selected depending on the intended purpose. Examples of the binding agent include water, ethanol, propanol, simple syrup, glucose liquid, starch liquid, gelatine liquid, carboxymethylcellulose, hydroxypropylcellulose, hydroxypropylstarch, methylcellulose, ethylcellulose, shellac, calcium phosphate and polyvinylpyrrolidone.

The disintegrating agent is not particularly limited and may be appropriately selected depending on the intended purpose. Examples of the disintegrating agent include dry starch, sodium alginate, powdered agar, sodium hydrogencarbonate, calcium carbonate, sodium lauryl sulfate, monoglyceride stearate and lactose.

The lubricating agent is not particularly limited and may be appropriately selected depending on the intended purpose. Examples of the lubricating agent include purified talc, stearic acid salts, borax and polyethylene glycol.

The coloring agent is not particularly limited and may be appropriately selected depending on the intended purpose. Examples of the coloring agent include titanium oxide and iron oxide.

The sweetening/flavoring agent is not particularly limited and may be appropriately selected depending on the intended purpose. Examples of the sweetening/flavoring agent include sucrose, orange peel, citric acid and tartaric acid.

### —Oral Semisolid Preparation—

The oral semisolid preparation is not particularly limited and may be appropriately selected depending on the intended purpose so long as it has intermediate properties between a liquid preparation and a solid preparation. Examples thereof include electuary, a chewing gum preparation, a whip preparation and a jelly preparation.

The method for producing the oral semisolid preparation is not particularly limited and may be a routine method. For example, the oral semisolid preparation can be produced by adding a gelling agent, a thickening agent and a stabilizing agent to at least one of a mixture of Ingredients (A) and (B) and a mixture of Ingredients (C) and (D).

The gelling agent is not particularly limited and may be appropriately selected depending on the intended purpose. Examples thereof include agar, gelatin, starch and gellan.

The thickening agent is not particularly limited and may be appropriately selected depending on the intended purpose. Examples thereof include xanthan, carrageenan, locust, guar, tamarind and pectin.

The stabilizing agent is not particularly limited and may be appropriately selected depending on the intended purpose. Examples thereof include tragacanth, gum arabic and gum ghatti.

### —Oral Liquid Preparation—

The oral liquid preparation is not particularly limited and may be appropriately selected depending on the intended purpose. Examples thereof include an internal liquid preparation, syrup and elixir.

The method for producing the oral liquid preparation is not particularly limited and may be a routine method. For example, the oral liquid preparation can be produced by adding an additive to at least one of a mixture of Ingredients (A) and (B) and a mixture of Ingredients (C) and (D).

The additive is not particularly limited and may be appropriately selected depending on the intended purpose. Examples thereof include a sweetening/flavoring agent, a buffer and a stabilizing agent.

The sweetening/flavoring agent is not particularly limited and may be appropriately selected depending on the intended purpose. Examples thereof include sucrose, orange peel, citric acid and tartaric acid.

The buffer is not particularly limited and may be appropriately selected depending on the intended purpose. Examples thereof include sodium citrate.

The stabilizing agent is not particularly limited and may be appropriately selected depending on the intended purpose. Examples thereof include tragacanth, gum arabic and gelatin.

### <Intake>

The method, amount, time and target of intake of the composition are not particularly limited and may be appropriately selected depending on the intended purpose.

The amount of intake thereof is not particularly limited and may be appropriately determined considering various factors of target individuals such as their age, body weight, constitution, symptoms and concomitant use of a drug containing other active ingredients.

Regarding animal species that can be targets of intake thereof, the composition is suitably applied to human. However, so long as the effects of the composition can be obtained, the composition may also be applied to non-human animals such as mice, rats, hamsters, birds, dogs, cats, sheep, goats, bovine, pigs and monkeys.

### <Application>

The composition can be used for a food or beverage having physiological activities such as an anti-cancer effect, an anti-inflammatory effect and a sugar metabolism-regulating effect.

### EXAMPLES

The present invention will next be described in more detail by way of Examples, which should not be construed as limiting the present invention thereto.

### Comparative Examples 1 and 2

### Stabilities of PPT (Ingredient (B)) and PPD (Ingredient (D))

### <Method>

5 mg of protopanaxatriol (PPT) powder (Ingredient (B)) (product of LKT Laboratories Inc.) (Comparative Example 1) and 5 mg of protopanaxadiol (PPD) powder (Ingredient (D)) (product of LKT Laboratories Inc.) (Comparative Example 2) were separately added to brown bottles with screw caps and left to stand still for 3 weeks under conditions of 40° C. and relative humidity 75%. After that, about 1 mg of each of the protopanaxatriol (PPT) powder (Ingredient (B)) and the protopanaxadiol (PPD) powder (Ingredient (D)) was dissolved in 1 mL of ethanol for high-performance liquid chromatography (purity: 99.5% by mass) (product of Wako Pure Chemical Industries, Ltd.) and analyzed through high-performance liquid chromatography under the following conditions.

### —Analysis Conditions—

Apparatus: 1200 series high-performance liquid chromatograph (product of Agilent Technologies, Ltd.)
Column: TSKgel ODS-80Ts, inner diameter: 4.6 mm, length: 15 cm (product of TOSOH CORPORATION, Ltd.)
Column temperature: 40° C.
Eluent water: acetonitrile=50:50 (for analysis of PPT)

water: acetonitrile=30:70 (for analysis of PPD)

Measurement wavelength: 196 nm
Flow rate: 1 mL/min
Injection amount: 10 μL

### <Results>

As a result of the above analysis, neither the protopanaxatriol (PPT) powder (Ingredient (B)) (Comparative Example 1) nor the protopanaxadiol (PPD) powder (Ingredient (D)) (Comparative Example 2) was detected. These results indicate that the protopanaxatriol (PPT) powder (Ingredient (B)) and the protopanaxadiol (PPD) powder (Ingredient (D)) are very poor in stability when they are left to stand still alone.

### Examples 1 to 3 and Comparative Examples 3 to 7

### Mixing Ratio of PT (Ingredient (A)) and PPT (Ingredient (B)) Used in Combination and Stability of PPT (Ingredient (B))

Panaxatriol (PT) powder (Ingredient (A)) (product of LKT Laboratories Inc.) and protopanaxatriol (PPT) powder (Ingredient (B)) (product of LKT Laboratories Inc.) were mixed together at mixing ratios as shown in Table 1 so that the total mass thereof was 10 mg. Each of the resultant mixtures was added to a brown bottle with a screw cap and left to stand still for one month under conditions of 40° C. and relative humidity 75%. After that, about 10 mg of the mixture of the panaxatriol (PT) powder (Ingredient (A)) and the protopanaxatriol (PPT) powder (Ingredient (B)), which had been left to stand still under the above conditions, was dissolved in 1 mL of ethanol for high-performance liquid chromatography (purity: 99.5% by mass) (product of Wako Pure Chemical Industries, Ltd.). The resultant ethanol solution was analyzed through high-performance liquid chromatography under the same analysis conditions as in Comparative Examples 1 and 2, to thereby quantify the amounts of panaxatriol (PT) (Ingredient (A)) and protopanaxatriol (PPT) (Ingredient (B)). The results are shown in Table 1.

TABLE 1

| | Comparative Examples | | | | | Examples | | |
|---|---|---|---|---|---|---|---|---|
| | 3 | 4 | 5 | 6 | 7 | 1 | 2 | 3 |
| (A) Amount of PT | 0.1 | 1 | 2 | 3 | 4 | 5 | 9 | 9.5 |
| (mg) | | | | | | | | |
| (B) Amount of PPT | 9.9 | 9 | 8 | 7 | 6 | 5 | 1 | 0.5 |
| (mg) | | | | | | | | |
| (A) PT/(B) PPT | 1/99 | 10/90 | 20/80 | 30/70 | 40/60 | 50/50 | 90/10 | 95/5 |
| (ratio by mass) | 0.01 | 0.11 | 0.25 | 0.43 | 0.67 | 1 | 9 | 19 |
| (A) Residual rate of PT | 98 | 98 | 97 | 100 | 101 | 99 | 101 | 98 |
| (% by mass) | | | | | | | | |
| (B) Residual rate of PPT | 0 | 4 | 11 | 32 | 72 | 92 | 95 | 100 |
| (% by mass) | | | | | | | | |

### <Results>

As is clear from Table 1, it is confirmed that the ratio by mass of (A) PT/(B) PPT is preferably adjusted to 50/50 (=1) or greater, more preferably 90/10 (=9) or greater, still more preferably 95/5 (=19) or greater, in order to keep the amount of PPT for one month under conditions of 40° C. and relative humidity 75%.

### Examples 4 to 6 and Comparative Examples 8 to 12

### Mixing Ratio of PD (Ingredient (C)) and PPD (Ingredient (D)) Used in Combination and Stability of PPD (Ingredient (D))

Panaxadiol (PD) powder (Ingredient (C)) (product of LKT Laboratories Inc.) and protopanaxadiol (PPD) powder (Ingredient (D)) (product of LKT Laboratories Inc.) were mixed together at mixing ratios as shown in Table 2 so that the total mass thereof was 10 mg. Each of the resultant mixtures was added to a brown bottle with a screw cap and left to stand still for one month under conditions of 40° C. and relative humidity 75%. After that, about 10 mg of the mixture of the panaxadiol (PD) powder (Ingredient (C)) and the protopanaxadiol (PPD) powder (Ingredient (D)), which had been left to stand still under the above conditions, was dissolved in 1 mL of ethanol for high-performance liquid chromatography (purity: 99.5% by mass) (product of Wako Pure Chemical Industries, Ltd.). The resultant ethanol solution was analyzed through high-performance liquid chromatography under the same analysis conditions as in Comparative Examples 1 and 2, to thereby quantify the amounts of panaxadiol (PD) (Ingredient (C)) and protopanaxadiol (PPD) (Ingredient (D)). The results are shown in Table 2.

TABLE 2

| | Comparative Examples | | | | | Examples | | |
|---|---|---|---|---|---|---|---|---|
| | 8 | 9 | 10 | 11 | 12 | 4 | 5 | 6 |
| (C) Amount of PD | 0.1 | 1 | 2 | 3 | 4 | 5 | 9 | 9.5 |
| (mg) | | | | | | | | |
| (D) Amount of PPD | 9.9 | 9 | 8 | 7 | 6 | 5 | 1 | 0.5 |
| (mg) | | | | | | | | |
| (C) PD/(D) PPD | 1/99 | 10/90 | 20/80 | 30/70 | 40/60 | 50/50 | 90/10 | 95/5 |
| (ratio by mass) | 0.01 | 0.11 | 0.25 | 0.43 | 0.67 | 1 | 9 | 19 |
| (C) Residual rate of PD | 98 | 99 | 100 | 100 | 99 | 101 | 100 | 101 |
| (% by mass) | | | | | | | | |
| (D) Residual rate of PPD | 0 | 6 | 14 | 41 | 75 | 93 | 95 | 100 |
| (% by mass) | | | | | | | | |

### <Results>

As is clear from Table 2, it is confirmed that the ratio by mass of (C) PD/(D) PPD is preferably adjusted to 50/50 (=1) or greater, more preferably 90/10 (=9) or greater, still more preferably 95/5 (=19) or greater, in order to keep the amount of PPD for one month under conditions of 40° C. and relative humidity 75%.

### Examples 7 to 15 and Comparative Examples 13 to 27

### Stability of PPT (Ingredient (B)) Used in Combination with Pt (Ingredient (A)) in an Ethanol Solution

### <Method>

10 mg of panaxatriol (PT) (Ingredient (A)) (product of LKT Laboratories, Inc.) and 10 mg of protopanaxatriol (PPT) (Ingredient (B)) (product of LKT Laboratories, Inc.) were each dissolved in 0.5 mL of ethanol. The pH of each of the solutions was adjusted to 3.5, 6.8 or 8.3 with 5% by mass hydrochloric acid or 1M aqueous sodium hydroxide. The PT ethanol solution and the PPT ethanol solution, whose pH had been adjusted to each of these values, were mixed together with appropriately diluted with ethanol, to thereby prepare ethanol solutions having PT and PPT concentrations shown in Tables 3 to 5. Each of the thus-prepared ethanol solutions was added to a brown bottle with a screw cap and left to stand still for one month under conditions of 40° C. and relative humidity 75%. After that, the panaxatriol (PT) (Ingredient (A)) ethanol solution and the protopanaxatriol (PPT) (Ingredient (B)) ethanol solution, which had been left to stand still under the above conditions, were 10-fold diluted with ethanol. The diluted ethanol solutions were analyzed through high-performance liquid chromatography under the same analysis conditions as in Comparative Examples 1 and 2, to thereby analyze the amount of the protopanaxatriol (PPT) (Ingredient (B)). The obtained value was used to calculate a residual rate (% by mass) of the PPT relative to the initial amount thereof. The results are shown in Tables 3 to 5.

TABLE 3

| | Comparative Examples | | | | | Examples | | |
|---|---|---|---|---|---|---|---|---|
| | 13 | 14 | 15 | 16 | 17 | 7 | 8 | 9 |
| pH | 3.5 | 3.5 | 3.5 | 3.5 | 3.5 | 3.5 | 3.5 | 3.5 |
| (A) Amount of | 0.01 | 0.1 | 0.2 | 0.3 | 0.4 | 0.5 | 0.9 | 0.95 |
| PT (% by | | | | | | | | |
| mass) | | | | | | | | |
| (B) Amount of | 0.99 | 0.9 | 0.8 | 0.7 | 0.6 | 0.5 | 0.1 | 0.05 |
| PPT (% by | | | | | | | | |
| mass) | | | | | | | | |
| (A) PT/(B) | 1/99 | 10/90 | 20/80 | 30/70 | 40/60 | 50/50 | 90/10 | 95/5 |
| PPT (ratio by | 0.01 | 0.11 | 0.25 | 0.43 | 0.67 | 1 | 9 | 19 |
| mass) | | | | | | | | |
| (B) Residual | 0 | 0 | 0.4 | 25.8 | 58.6 | 90.2 | 91.4 | 94.7 |
| rate of PPT | | | | | | | | |
| (% by mass) | | | | | | | | |

TABLE 4

| | Comparative Examples | | | | | Examples | | |
|---|---|---|---|---|---|---|---|---|
| | 18 | 19 | 20 | 21 | 22 | 10 | 11 | 12 |
| pH | 6.8 | 6.8 | 6.8 | 6.8 | 6.8 | 6.8 | 6.8 | 6.8 |
| (A) Amount of | 0.01 | 0.1 | 0.2 | 0.3 | 0.4 | 0.5 | 0.9 | 0.95 |
| PT (% by | | | | | | | | |
| mass) | | | | | | | | |
| (B) Amount of | 0.99 | 0.9 | 0.8 | 0.7 | 0.6 | 0.5 | 0.1 | 0.05 |
| PPT (% by | | | | | | | | |
| mass) | | | | | | | | |
| (A) PT/(B) | 1/99 | 10/90 | 20/80 | 30/70 | 40/60 | 50/50 | 90/10 | 95/5 |
| PPT (ratio by | 0.01 | 0.11 | 0.25 | 0.43 | 0.67 | 1 | 9 | 19 |
| mass) | | | | | | | | |
| (B) Residual | 0 | 0.1 | 0.6 | 27.8 | 60.2 | 91.6 | 94.2 | 98.7 |
| rate of PPT | | | | | | | | |
| (% by mass) | | | | | | | | |

TABLE 5

| | Comparative Examples | | | | | Examples | | |
|---|---|---|---|---|---|---|---|---|
| | 23 | 24 | 25 | 26 | 27 | 13 | 14 | 15 |
| pH | 8.3 | 8.3 | 8.3 | 8.3 | 8.3 | 8.3 | 8.3 | 8.3 |
| (A) Amount of | 0.01 | 0.1 | 0.2 | 0.3 | 0.4 | 0.5 | 0.9 | 0.95 |
| PT (% by | | | | | | | | |
| mass) | | | | | | | | |
| (B) Amount of | 0.99 | 0.9 | 0.8 | 0.7 | 0.6 | 0.5 | 0.1 | 0.05 |
| PPT (% by | | | | | | | | |
| mass) | | | | | | | | |
| (A) PT/(B) | 1/99 | 10/90 | 20/80 | 30/70 | 40/60 | 50/50 | 90/10 | 95/5 |
| PPT (ratio by | 0.01 | 0.11 | 0.25 | 0.43 | 0.67 | 1 | 9 | 19 |
| mass) | | | | | | | | |
| (B) Residual | 0 | 0.8 | 1.4 | 30.9 | 62.8 | 91.4 | 93.4 | 97.1 |
| rate of PPT | | | | | | | | |
| (% by mass) | | | | | | | | |

### <Results>

As is clear from Tables 3 to 5, when the ratio by mass of (A) PT/(B) PPT was smaller than 50/50 (=1), the stability of the PPT (Ingredient (B)) was poor (Comparative Examples 13 to 27). Especially when the pH of the ethanol solutions was lower, the residual rate of the PPT (Ingredient (B)) was lower (Comparative Examples 13 to 17). Meanwhile, when the ratio by mass of (A) PT/(B) PPT was 50/50 (=1) or greater, the residual rate of the PPT was 90% by mass or greater in all the ethanol solutions regardless of the pH thereof (Examples 7 to 15). It was confirmed that the stability of the PPT (Ingredient (B)) was kept even at the low pH (Examples 7 to 9).

These results confirm that the ratio by mass of (A) PT/(B) PPT is preferably adjusted to 50/50 (=1) or greater, more preferably 90/10 (=9) or greater, still more preferably 95/5 (=19) or greater, in order to keep the amount of PPT for one month.

### Examples 16 to 24 and Comparative Examples 28 to 42

### Stability of PPD (Ingredient (D)) Used in Combination with Pd (Ingredient (C)) in an Ethanol Solution)

### <Method>

10 mg of panaxadiol (PD) (Ingredient (C)) (product of LKT Laboratories, Inc.) and 10 mg of protopanaxadiol (PPD) (Ingredient (D)) (product of LKT Laboratories, Inc.) were each dissolved in 0.5 mL of ethanol. The pH of each of the solutions was adjusted to 3.5, 6.8 or 8.3 with 5% by mass hydrochloric acid or 1M aqueous sodium hydroxide. The PD ethanol solution and the PPD ethanol solution, whose pH had been adjusted to each of these values, were mixed together with appropriately diluted with ethanol, to thereby prepare ethanol solutions having PD and PPD concentrations shown in Tables 6 to 8. Each of the thus-prepared ethanol solutions was added to a brown bottle with a screw cap and left to stand still for one month under conditions of 40° C. and relative humidity 75%. After that, the protopanaxadiol (PPD) (Ingredient (D)) ethanol solution and the panaxadiol (PD) (Ingredient (C)) ethanol solution, which had been left to stand still under the above conditions, were 10-fold diluted with ethanol. The diluted ethanol solutions were analyzed through high-performance liquid chromatography under the same analysis conditions as in Comparative Examples 1 and 2, to thereby analyze the amount of the protopanaxadiol (PPD) (Ingredient (D)). The obtained value was used to calculate a residual rate (% by mass) of the PPD relative to the initial amount thereof. The results are shown in Tables 6 to 8.

TABLE 6

| | Comparative Examples | | | | | Examples | | |
|---|---|---|---|---|---|---|---|---|
| | 28 | 29 | 30 | 31 | 32 | 16 | 17 | 18 |
| pH | 3.5 | 3.5 | 3.5 | 3.5 | 3.5 | 3.5 | 3.5 | 3.5 |
| (C) Amount of | 0.01 | 0.1 | 0.2 | 0.3 | 0.4 | 0.5 | 0.9 | 0.95 |
| PD (% by | | | | | | | | |
| mass) | | | | | | | | |
| (D) Amount of | 0.99 | 0.9 | 0.8 | 0.7 | 0.6 | 0.5 | 0.1 | 0.05 |
| PPD (% by | | | | | | | | |
| mass) | | | | | | | | |
| (C) PD/(D) | 1/99 | 10/90 | 20/80 | 30/70 | 40/60 | 50/50 | 90/10 | 95/5 |
| PPD (ratio by | 0.01 | 0.11 | 0.25 | 0.43 | 0.67 | 1 | 9 | 19 |
| mass) | | | | | | | | |
| (D) Residual | 0 | 0 | 0.8 | 23.5 | 58.5 | 90.1 | 90.8 | 94.8 |
| rate of PPD | | | | | | | | |
| (% by mass) | | | | | | | | |

TABLE 7

| | Comparative Examples | | | | | Examples | | |
|---|---|---|---|---|---|---|---|---|
| | 33 | 34 | 35 | 36 | 37 | 19 | 20 | 21 |
| pH | 6.8 | 6.8 | 6.8 | 6.8 | 6.8 | 6.8 | 6.8 | 6.8 |
| (C) Amount of | 0.01 | 0.1 | 0.2 | 0.3 | 0.4 | 0.5 | 0.9 | 0.95 |
| PD (% by | | | | | | | | |
| mass) | | | | | | | | |
| (D) Amount of | 0.99 | 0.9 | 0.8 | 0.7 | 0.6 | 0.5 | 0.1 | 0.05 |
| PPD (% by | | | | | | | | |
| mass) | | | | | | | | |
| (C) PD/(D) | 1/99 | 10/90 | 20/80 | 30/70 | 40/60 | 50/50 | 90/10 | 95/5 |
| PPD (ratio by | 0.01 | 0.11 | 0.25 | 0.43 | 0.67 | 1 | 9 | 19 |
| mass) | | | | | | | | |
| (D) Residual | 0.5 | 3.0 | 6.3 | 40.6 | 69.5 | 91.2 | 95.7 | 99.8 |
| rate of PPD | | | | | | | | |
| (% by mass) | | | | | | | | |

TABLE 8

| | Comparative Examples | | | | | Examples | | |
|---|---|---|---|---|---|---|---|---|
| | 38 | 39 | 40 | 41 | 42 | 22 | 23 | 24 |
| pH | 8.3 | 8.3 | 8.3 | 8.3 | 8.3 | 8.3 | 8.3 | 8.3 |
| (C) Amount of | 0.01 | 0.1 | 0.2 | 0.3 | 0.4 | 0.5 | 0.9 | 0.95 |
| PD (% by | | | | | | | | |
| mass) | | | | | | | | |
| (D) Amount of | 0.99 | 0.9 | 0.8 | 0.7 | 0.6 | 0.5 | 0.1 | 0.05 |
| PPD (% by | | | | | | | | |
| mass) | | | | | | | | |
| (C) PD/(D) | 1/99 | 10/90 | 20/80 | 30/70 | 40/60 | 50/50 | 90/10 | 95/5 |
| PPD (ratio by | 0.01 | 0.11 | 0.25 | 0.43 | 0.67 | 1 | 9 | 19 |
| mass) | | | | | | | | |
| (D) Residual | 0 | 0.5 | 1.8 | 43.5 | 74.3 | 90.8 | 94.7 | 98.8 |
| rate of PPD | | | | | | | | |
| (% by mass) | | | | | | | | |

### <Results>

As is clear from Tables 6 to 8, when the ratio by mass of (C) PD/(D) PPD was smaller than 50/50 (=1), the stability of the PPD (Ingredient (D)) was poor (Comparative Examples 28 to 42). Especially when the pH of the ethanol solutions was lower, the residual rate of the PPD (Ingredient (D)) was lower (Comparative Examples 28 to 32). Meanwhile, when the ratio by mass of (C) PD/(D) PPD was 50/50 (=1) or greater, the residual rate of the PPD was 90% by mass or greater in all the ethanol solutions regardless of the pH thereof (Examples 16 to 24). It was confirmed that the stability of the PPD (Ingredient (D)) was kept even at the low pH (Examples 16 to 18).

These results confirm that the ratio by mass of (C) PD/(D) PPD is preferably adjusted to 50/50 (=1) or greater, more preferably 90/10 (=9) or greater, still more preferably 95/5 (=19) or greater, in order to keep the amount of PPD for one month.

Although protopanaxatriol (PPT) (Ingredient (B)) and protopanaxadiol (PPD) (Ingredient (D)) have excellent physiological activities such as an anti-cancer effect, an anti-inflammatory effect and a sugar metabolism-regulating effect, their stability is poor to thereby make it difficult to provide a composition containing a large amount of PPT (Ingredient (B)) and PPD (Ingredient (D)). However, as is understood from the results of Examples 1 to 24, it is possible to provide a composition containing a large amount of PPT (Ingredient (B)) and PPD (Ingredient (D)).

The composition of the present invention contains a large amount of at least one of protopanaxatriol (PPT) and protopanaxadiol (PPD) and also has high stability. Thus, it can be used as a composition capable of stably exhibiting excellent physiological activities such as an anti-cancer effect, an anti-inflammatory effect and a sugar metabolism-regulating effect. The composition can be suitably used as a food or beverage as well.

Aspects of the present invention are as follows, for example.

<1> A composition including:

at least one mixture selected from the group consisting of a mixture of (A) panaxatriol and (B) protopanaxatriol and a mixture of (C) panaxadiol and (D) protopanaxadiol, wherein a ratio (A)/(B) of a mass of the (A) panaxatriol to a mass of the (B) protopanaxatriol is 1 or greater, and a ratio (C)/(D) of a mass of the (C) panaxadiol to a mass of the (D) protopanaxadiol is 1 or greater.

<2> The composition according to <1>, wherein at least one of the (B) protopanaxatriol and the (D) protopanaxadiol is in the form of powder, and after the composition has been stand still for one month under conditions of 40° C. and relative humidity 75%, a residual rate of the at least one of the (B) protopanaxatriol and the (D) protopanaxadiol is 90% by mass or more.

<3> The composition according to <1>, wherein at least one of the (B) protopanaxatriol and the (D) protopanaxadiol is in the form of ethanol solution, and after the composition has been stand still for one month under conditions of 40° C. and relative humidity 75%, a residual rate of the at least one of the (B) protopanaxatriol and the (D) protopanaxadiol is 90% by mass or more.

<4> The composition according to any one of <1> to <3>, wherein the composition is a food or beverage.

What is claimed is:

1. A composition comprising:
at least one mixture selected from the group consisting of a mixture of (A) panaxatriol and (B) protopanaxatriol and a mixture of (C) panaxadiol and (D) protopanaxadiol,
wherein a ratio (A)/(B) of a mass of the (A) panaxatriol to a mass of the (B) protopanaxatriol is 1 or greater, and a ratio (C)/(D) of a mass of the (C) panaxadiol to a mass of the (D) protopanaxadiol is 1 or greater.

2. The composition according to claim 1, wherein the composition is a food or beverage.
